(19) United States
(12) Patent Application Publication
(10) Pub. No.: US 2024/0197936 A1
(43) Pub. Date: Jun. 20, 2024

(54) A DEVICE WITH CUSTOMIZED INTEGRATED ELECTRONIC CIRCUIT FOR DESTROYING PATHOGENS

(71) Applicant: NarsimhaChary Mandaji, Hyderabad (IN)

(72) Inventor: NarsimhaChary Mandaji, Hyderabad (IN)

(73) Assignee: NarsimhaChary Mandaji, Hyderabad (IN)

(21) Appl. No.: 18/555,256

(22) PCT Filed: Jun. 15, 2022
(86) PCT No.: PCT/IN2022/050548

§ 371 Date: Oct. 13, 2023

(30) Foreign Application Priority Data
Jun. 17, 2021 (IN) 202141027063

(51) Int. Cl. A61L 2/10; A61L 9/20; H05B 41/288; H05B 41/292

(52) U.S. Cl. CPC A61L 2/10; A61L 9/20; H05B 41/288; H05B 41/2926; A61L 2202/11; A61L 2209/12

(57) ABSTRACT

The present invention is related to a device for generating high lux intensity light comprising a filament-less lamp with a customized integrated electronic circuit that can maximize the lux intensity. The customized integrated electronic circuit has also been used in a transformer-less and battery-less high-density electron generator for generating high electron acceleration. The low-cost, light weight device is effective in killing harmful pathogens in a very short span of time.

### CROSS-REFERENCE TO RELATED APPLICATIONS

This application claims the benefit of, and is a continuation of an International Application Number PCT/IN2022/050548, filed Jun. 15, 2022, which claims the benefit of foreign priority application number 202141027063, filed in India on Jun. 17, 2021, issued as Indian patent number 430863. The contents of the foregoing applications are incorporated herein by reference.

### FIELD OF INVENTION

The present invention is related to a device for inactivating or killing pathogens, wherein the device comprises a filament-less lamp with customized integrated electronic circuit. The present invention is also related to a device for inactivating or killing pathogens, wherein the device comprises a transformer-less and battery-less electron beam generator with customized integrated electronic circuit.

The present invention also relates to a method for inactivating or killing the pathogens and use of a device for the same.

### BACKGROUND OF THE INVENTION

Ultraviolet energy is generally identified by its wavelength. The wavelength value is measured in nanometers (nm) which determines the type of ultraviolet energy. UV-A encompasses wavelength between 315 and 400 nanometers, UV-B wavelength falls between at 280 and 315 nanometers, and UV-C wavelength falls between 100 and 280 nanometers. UV-C wavelength is effective in inactivating pathogens including viruses, bacteria, molds and fungi.

UV-C is an effective germicidal wavelength because DNA and RNA are susceptible to damage around 265 nanometers. When pathogens are exposed to UV-C wavelength radiation, double bonds that tie thymine and adenine together are destroyed in a process called dimerization, altering the structure of the pathogen's genome. Due to this alteration, when the pathogen attempts to replicate or reproduce, the genomic alteration prevents it from doing so successfully.

UV-C is unique in its ability to perform germicidal functions because of the wavelength susceptibility of thymine (uracil in RNA). Ultraviolet-C(UV-C) lamp is commonly used on surfaces to reduce certain viruses and bacteria responsible for illness. However, different inventors have followed different approaches for killing harmful pathogens.

CN1303364C discloses a microwave electrodeless discharging UV ray disinfectant microwave oven capable of utilizing microwave excitation to produce UV rays that perform disinfection. It uses power attenuator for regulating and controlling microwave power.

U.S. Pat. No. 9,339,579B2 discloses a contamination control system for ductless air conditioners. The contamination control system comprises decontamination lamps contained within a tubular housing. The decontamination lamp may be activated with a photoactivated sensor. The decontamination lamps enclosed in a tubular housing may be a shielded UV lamp with less than ½″ diameter capable of emitting germicidal UV light.

EP650312B1 discloses a transformer-less ballast circuit which ionizes the gas present within a lamp without the utilization of filaments or inductive components.

JP2002209982A discloses an UV ray sterilizer using a magnetron and electrodeless lamp for sterilization of shoes.

CN212699749U discloses a microwave electrodeless UV disinfection cabinet for household appliances.

CN104616964A discloses a cold cathode dimming non-filament light source. It is difficult to achieve a wide range of voltages with the use of high power devices.

U.S. Pat. No. 6,953,940B2 discloses a hand-held germicidal UV lamp with child safety features to sanitize portable, compact and small objects or equipment such as toothbrushes, dental tools, etc.

ASHRAE validates effectiveness of UV-C germicidal irradiation (UVGI) tube light that inactivates microorganisms with a kill ratio of 90% or higher.

Steril-Aire clean comfort single lamp UV coil purifier is suitable for residential air handler installations.

The Honeywell UV treatment system is a portable UV-C lamp that irradiates entire cross-section of passenger seating using high intensity 254 nm UV-C lamp. It can treat a cabin or coach of thirty rows of seating within ten minutes.

UV-C technology has been found to significantly reduce various bacteria and viruses on targeted surfaces at prescribed dosages. UV-C rays have the shortest wavelength and highest energy, which gives them the ability to destroy molecular bonds that hold together deoxyribonucleic acid (DNA) of viruses and bacteria. UV light is divided into three different classes: UV-A (315-400 nm), UV-B (280-315 nm), and UV-C (100-280 nm). DNA has maximum absorbency of UV light at 254 nm.

UV-C radiation has been shown to destroy the outer protein coating of the SARS-COV-2 virus. Most of the air sanitization devices face challenges with the design of the UV chamber with appropriate wavelength of UV lamp and intensified field strength of input drift electrons. The velocity of electrons is proportional to the mobility of electrons inside the UV assembly, with filament being the major challenge to be addressed.

Lux is a measure of the proportion of light falling on a surface. Attempt to increase the lux in the UV-C lamp, leads to possibility of bursting of the filaments. UV-C lamps available in the market have less lux, and when an attempt is made to increase the lux in the UV-C lamp, its filaments may break down leading to the failure of the lamp.

In order to overcome the above disadvantages, a customized integrated electronic circuit (also called Chary Formula) has been developed that could maximize lux intensity using the residual mercury until it becomes zero after diffusion of light. The current invention comprising a customized integrated electronic circuit can generate 1288 lux intensity, 1350 lux intensity at 80 ohms, 632 lux intensity at 160 ohms and 405 lux intensity at 240 ohms in the same UV-C lamp, by utilizing optimal mercury content which will increase the life span of the UV-C lamp. The current invention also comprises a customized integrated electronic circuit that can generate high density electrons to kill pathogens.

### OBJECT OF THE INVENTION

The principal object of the present invention is to design a customized integrated electronic circuit for a device that inactivates or kills pathogens including the SARS-CoV-2 virus and other infectious viruses within a few seconds which is many times faster than the other UV lamps available in the prior art, for disinfection.

Another object of the present invention is to provide a lightweight, low-cost device that can generate high lux intensity that can kill the pathogens in a very short span of time.

It is yet another object of the invention to provide a customized integrated electronic circuit that does not require a choke and starter in lamps, such as the filament-less UV lamps, compact fluorescent lamps (CFL), metal halide lamps, sodium vapor lamps, mercury vapor lamps, induction lamps, and hydrargyrum medium-arc iodide (HMI) lamps.

Still another object of the present invention is to provide a lightweight, low-cost device with high light-emitting efficiency, having long service life, ability to operate at low voltages, possessing simple circuitry, and also easy to manufacture and install.

Still yet another object of the invention is to provide a customized integrated electronic circuit along with a filament-less lamp that optimally utilizes the mercury content.

It is yet another object of the invention to provide a customized integrated electronic circuit that will maintain power factor near to unity.

It is yet another object of the invention to provide a device with an electron generator and customized integrated electronic circuit that generates high electron acceleration.

It is yet another object of the present invention to provide a lightweight, low-cost device that can generate high electron acceleration and can inactivate or kill the pathogens in a very short span of time while also being affordable to the common man.

It is yet another object of the present invention to provide a method of inactivating or killing pathogens in surrounding environment by using a filament-less UV-C lamp.

It is yet another object of the present invention to provide a method of inactivating or killing pathogens in surrounding environment by using a device that is capable of generating high electron acceleration.

### BRIEF SUMMARY OF THE INVENTION

The present invention is related to a filament-less lamp with a novel integrated electronic circuit.

The present invention is also related to a UV-C filament-less lamp.

The present invention further relates to a device that is capable of enhancing the velocity and density of generated electrons from a novel integrated electronic circuit.

Accordingly, the present invention is related to a device for generating high lux intensity light comprising a filament-less lamp with a customized integrated electronic circuit comprising a filament-less lamp, plurality of diodes, plurality of resistors, a variable resistor, capacitors, and a strip attached to the filament-less lamp. The combination of plurality of diodes and plurality of resistors forms a DC bridge rectifier circuit. The combination of plurality of diodes and capacitors forms a voltage multiplier circuit. The voltage multiplier circuit enables the conversion of 230 V AC to 650-700 V DC. The positive terminal of the voltage multiplier circuit is connected to one end of the filament-less lamp through the variable resistor. The negative terminal of the voltage multiplier circuit is connected to another end of the filament-less lamp. The strip attached to the filament-less lamp is made up of silver, copper, gold, aluminum, iron or a combination thereof.

The number of resistors can be selected according to the end utility of the present invention. Accordingly, the present invention also defines a device for generating high lux intensity light comprising a filament-less lamp with a customized integrated electronic circuit comprising a filament-less lamp, plurality of diodes, plurality of resistors, a variable resistor, capacitors, and a strip attached to the filament-less lamp.

The present invention further relates to a device for generating high density electrons comprising a transformer-less and battery-less generator with a customized integrated electronic circuit comprising plurality of diodes, plurality of resistors, a variable resistor, capacitors, Zener diodes, a transistor (TIP 41C), and one or more copper coils. The combination of plurality of diodes and plurality of resistors forms a DC bridge rectifier circuit. The combination of plurality of diodes and capacitors forms a voltage multiplier circuit.

In another embodiment, the present invention also provides a transformer-less and battery-less high density electron generator with a customized integrated electronic circuit along with a cooling fan.

The present invention also relates to a method of inactivating or killing pathogens in surrounding environment by using any of the devices of the present invention.

The present invention also relates to the use of a device in accordance with the various embodiments, for inactivating or killing pathogens in surrounding environment.

### BRIEF DESCRIPTION OF THE DRAWINGS

The summary of the present invention, as well as the detailed description, is better understood when read in conjunction with the accompanying figures, wherein, FIG. 1a illustrates a filament-less lamp device comprising a filament-less lamp with a customized integrated electronic circuit comprising of plurality of diodes, plurality of resistors, a variable resistor and capacitors for generating high lux intensity, in accordance with the present invention;

FIG. 1b illustrates a filament-less lamp device comprising a filament-less lamp with a customized integrated electronic circuit comprising of diodes (D1, D2, D3, and D4), resistors (R1, R2, R3, R4, R6, R7, and R8), a variable resistor (R5), capacitors (C1, C2) and a strip attached to the filament-less lamp, wherein a combination of diodes (D1, D2, D3, and D4) and resistors (R1, R2, R3, and R4) forms DC bridge rectifier circuit, and combination of diodes (D1, D2, D3, and D4) and capacitors (C1, C2) forms a voltage multiplier circuit, in accordance with the present invention;

FIG. 1c illustrates a filament-less lamp device comprising a filament-less lamp with a modified integrated electronic circuit comprising of diodes (D1, D2, D3, and D4), resistors (R1, R2, R3, and R4), a variable resistor (R1), capacitors (C1, C2), and a strip attached to the filament-less lamp for generating high lux intensity, in accordance with the present invention;

FIG. 2a illustrates a transformer-less and battery-less generator with a customized integrated electronic circuit comprising plurality of diodes, plurality of resistors, a variable resistor, capacitors. Zener diodes, a transistor (TIP 41C), and one or more copper coils; wherein, a combination of plurality of diodes, and plurality of resistors forms DC bridge rectifier circuit, and a combination of plurality of diodes and capacitors forms a voltage multiplier circuit, in accordance with the present invention;

FIG. 2b illustrates a device with transformer-less and battery-less high-density electron generator along with a customized integrated electronic circuit comprising diodes (D1, D2, D3, and D4), Zener diodes, resistors (R1, R2, R3, R4, R5, R6, R8 and R9), a variable resistor (R7), non-polarized capacitors (C1, C2, C3 and C4), a TIP 41C transistor, and copper coils, wherein a combination of diodes (D1, D2, D3, and D4) and resistors (R1, R2, R3, and R4) forms DC bridge rectifier circuit, and a combination of diodes (D1, D2, D3, and D4) and capacitors (C1, C2) forms a voltage multiplier circuit, in accordance with the present invention;

FIG. 2c illustrates a device with transformer-less and battery-less high-density electron generator along with a modified integrated electronic circuit comprising of diodes (D1, D2, D3 and D4). Zener diodes, resistors (R1, R2, R3, and R4), and capacitors (C1, C2, C3, C4), a transistor (TIP 41C), and copper coils, in accordance with the present invention;

FIG. 3 illustrates a device with transformer-less and battery-less high-density electron generator along with customized integrated electronic circuit comprising of diodes, Zener diodes, resistors, a variable resistor, non-polarized capacitors, a transistor (TIP 41C) and copper coils for generating high electron acceleration, along with a cooling fan, in accordance with the present invention;

FIG. 4a and FIG. 4b show viral reduction obtained using a device comprising a filament-less lamp with a customized integrated electronic circuit in accordance with the present invention; and

FIG. 5a and FIG. 5b show viral reduction obtained using a device comprising a transformer-less and battery-less generator with a customized integrated electronic circuit in accordance with the present invention.

### DETAILED DESCRIPTION OF THE INVENTION

The present invention relates to a device for generating high lux intensity comprising a filament-less lamp with a customized integrated electronic circuit, and a strip attached to the filament-less lamp.

The present invention also relates to a device for generating high density electrons comprising a transformer-less and battery-less generator with a customized integrated electronic circuit, Zener diodes, a transistor (TIP 41C), and one or more copper coils.

The present invention also relates to a method of inactivating and or killing the pathogens in surrounding environment by using any of the devices of the present invention.

The present invention also relates to use of a device for inactivating and or killing the pathogens in surrounding environment.

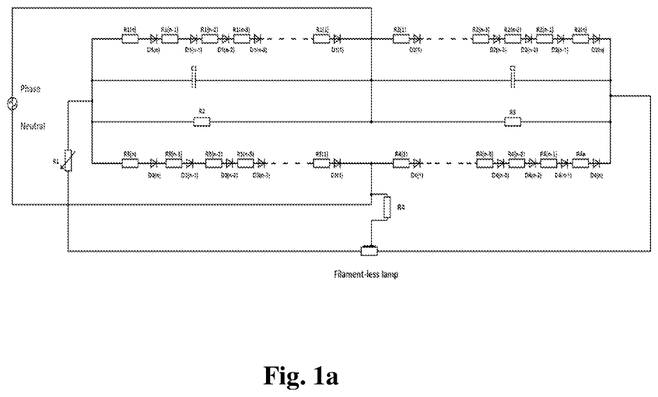

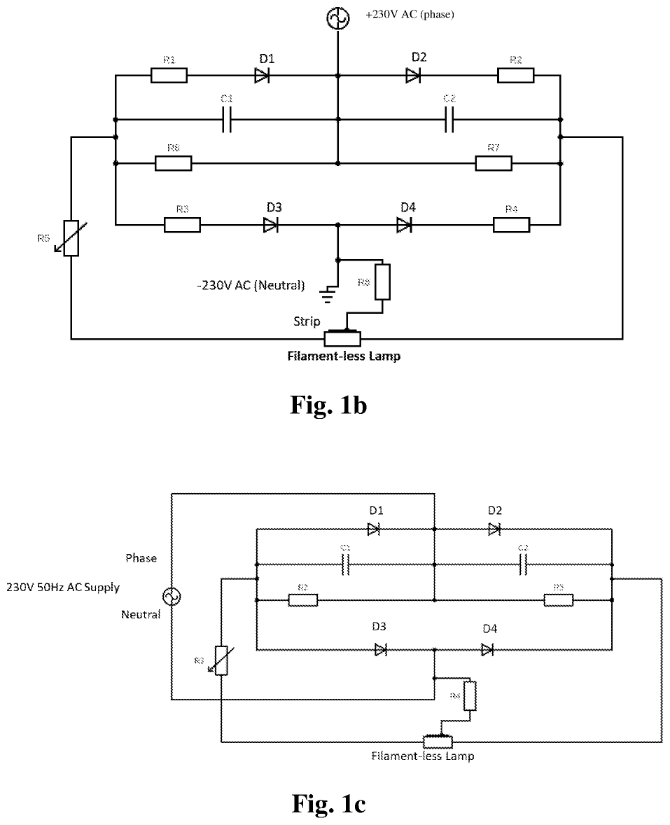

According to an embodiment of the present invention, as shown in FIG. 1a, a filament-less lamp device comprises a filament-less lamp with a customized integrated electronic circuit comprising of plurality of diodes, plurality of resistors, a variable resistor, capacitors, and a strip attached to the filament-less lamp, for generating high lux intensity. The combination of a plurality of diodes and plurality of resistors forms DC bridge rectifier circuit. The combination of plurality of diodes and capacitors forms a voltage multiplier circuit.

In another embodiment of the present invention, as shown in FIG. 1b, said device for generating high lux intensity light comprises a filament-less lamp with a customized integrated electronic circuit comprising a filament-less lamp, diodes (D1, D2, D3, and D4), resistors (R1, R2, R3, R4, R6, R7, and R8), a variable resistor (R5), capacitors (C1, C2), and a strip attached to the filament-less lamp. The combination of diodes (D1, D2, D3, and D4) and resistors (R1, R2, R3, and R4) forms a DC bridge rectifier circuit. The combination of diodes (D1, D2, D3, and D4) and capacitors (C1, C2) forms a voltage multiplier circuit. The voltage multiplier circuit enables the conversion of 230 V AC to 650-700 V DC. The positive terminal of the voltage multiplier circuit is connected to one end of the filament-less lamp through the variable resistor (R5). The negative terminal of the voltage multiplier circuit is connected to another end of the filament-less lamp. The strip attached to the filament-less lamp is made up of silver, copper, gold, aluminum, iron or a combination thereof.

Said resistors R1, R2, R3 and R4 have a resistance value ranging from 1 ohm to 3 ohms, and a power rating of 1000 watt to 2000 watts. Said resistors R6, R7 and R8 have a resistance value ranging from 1 mega ohm to 3 mega ohms and a power rating of 0.25-5 watts. The variable resistor (R5) has a resistance value ranging from 20 ohms to 100 ohms, and a power rating of 0.25 watts to 5 watts. The variable resistor R5 is made up of metals such as Nichrome, Iron, Copper, Aluminium, or a combination thereof. The non-polarized capacitor has a voltage rating ranging from 400 volts to 1050 volts.

A voltage multiplier circuit that is used to convert AC to high voltage DC comprises a combination of capacitors C1 and C2 and resistors R6 and R7, for use in said filament-less lamp for electron discharge phenomenon. The pulsating DC may change in its value but not in direction. The pulsating DC bridge rectifier circuit comprises diodes (D1, D2, D3, and D4) and resistors (R1, R2, R3, and R4). As the capacitor is connected to the output of a rectifier circuit, the capacitor charges to the peak rectified voltage during any half cycle, and the rectifier produces an output voltage. The capacitor then provides energy to the load until the rectifier produces another peak to charge the capacitor. Considering the total voltage across this path is 15 V=VcI+Vc2=Vm+Vm=2Vm, the effective capacitance of the circuit is Ci-C 2/Ci+C2=C.

A positive phase (230V) is coupled to diode D1 and diode D2 whereas the negative phase (230V) is coupled to diode D3 and diode D4. The non-polarized capacitors are connected in parallel to the resistors and diodes that help in the safe discharge of the capacitors C1, C2 when the power is off. One end of the non-polarized capacitor is connected to one end of the filament-less lamp through variable resistor R5, whereas the other end of the non-polarized capacitor is connected to the other end of the filament-less lamp or any other source of light including filament-less UV lamps, compact fluorescent lamps (CFL), metal halide lamps, sodium vapor lamps, mercury vapor lamps, induction lamps, and hydrargyrum medium-arc iodide (HMI) lamps.

The voltage multiplier circuit inputs DC voltage from the bridge rectifier and doubles the DC output voltage. The voltage multiplier circuit comprises four switching diodes D1, D2, D3 and D4 and non-polarized capacitors C1 and C2. The voltage multiplier circuit converts alternate current (AC) to direct current (DC) increasing the direct current (DC) level. When 230 V AC supply is provided, the voltage multiplier circuit enables conversion of AC to DC. 230 V AC is increased up to 650-700 V DC. The voltage multiplier circuit is used to convert AC voltage to high DC voltage which in the filament-less lamp chamber, creates an electron discharge phenomenon. The voltage multiplier circuit can be modified depending on the requirement of the application. High voltage multiplier circuits designed for the filament-less lamp chamber become the ultra-violet (UV) chamber. This ultra-violet (UV) chamber plays a vital role in electron acceleration that propels ultra-violet (UV) lux to very high speeds and energies, maintaining a well-defined wavelength beam in the ultra-violet (UV) chamber. The electrostatic acceleration causes static electric fields to accelerate photons resulting in high intensity and high quality ultra-violet (UV) lamp sources in the filament-less lamp chamber.

The electron drift in the filament-less lamp chamber has been optimized through the appropriate selection of resistors R1, R2, R3, and R4. The charged particles pass through the potential difference, and the output energy is limited by variable resistor R5. The acceleration of atomic nuclei is done using a variable resistor of metals such as Nichrome, Iron, Copper, Aluminium, or a combination thereof. The variable resistor plays a vital role in varying the particle beam parameters such as aspect ratio, current intensity and position of rays.

In the construction of said customized integrated electronic circuit, a variable resistor R5 is used for saving energy. The current consumption of equipment with a 0.5 power factor (as in the case of choke) is twice that of a load with a unity power factor (as in the case of the variable resistor). Therefore, to minimize the power factor, the variable resistor is employed. The charged particles pass through the potential difference, and the output energy is limited by the variable resistor (R5).

The most important requirement in the ultra-violet (UV) chamber is the volume of the ultra-violet (UV) field. When an electric field is applied, the electrons start drifting. If a high electric field is applied, then, electron drift accelerates in the ultra-violet (UV) chamber. This electron drifting is achieved by the appropriate selection of resistors R1, R2, R3, and R4. If there is an increase in field strength, then electrons emit UV light on the anode which is achieved by lowering the resistance values. The electrostatic force created by resistors R1, R2, R3, and R4 helps in moving the charged particles. Electron drift characterized by drift velocity is represented by the equation E=V/D, V_{d}(e)=Vμₑ/ρD, wherein E is the electric field, V is Voltage, D is the gap, He is electron mobility, p is the gas pressure. The electron mobility is increased by said customized integrated electronic circuit design presented in FIG. 1b. The free electrons are captured by the positive ions. The current in the customized integrated electronic circuit is balanced or limited by using variable resistor R5 shown in FIG. 1b. The resistance value decreases with an increase in the flow of electrons in the filament-less lamp. This causes emission of UV-C light of 254 nm on the anode.

Said customized integrated electronic circuit is coupled to the filament-less lamp. This filament-less lamp device has a rectangular strip of a material such as silver, copper, gold, aluminum, iron etc. over the glass tube body. The capacitor is a non-polarized capacitor of any type thereof.

In another embodiment, the present invention provides a filament-less lamp with a customized integrated electronic circuit comprising a filament-less lamp, a voltage multiplier circuit, resistors (R2, R3, and R4), a variable resistor (R1), and a strip as shown in FIG. 1c. Said device is capable of generating high lux intensity that can inactivate or kill dangerous pathogens in a very short time. The variable resistor R1 is intended to balance or limit the current in said customized integrated electronic circuit. The variable resistor R1 is made up of Nichrome, Iron, Copper, Aluminium or combination thereof. The strip is attached over a glass tube body. The bridge rectifier circuit comprises diodes (D1, D2, D3 and D4), resistors (R2, R3 and R4), and non-polarized capacitors (C1, C2).

The filament-less lamp generates a lux greater than 1200 that helps sanitize the entire area with 99% efficiency. It is maintained at a wavelength of 220-254 nm. The voltage multiplier circuit converts alternate current (AC) to direct current (DC) with increasing direct current (DC) level.

Said device comprising a filament-less lamp with a customized integrated electronic circuit which generates UV-C rays, sanitizes surrounding environment when placed on a surface, by inactivating or killing the viruses, bacteria and protozoa. The device of the present invention can inactivate or kill the SARS-COV-2 virus and other infectious viruses within seconds which is many times faster than other available UV-C lamps for disinfection.

Said device of the present invention is a lightweight, low cost, high efficiency light-emitting device with long service life. It has good applicability at low voltage, comprises a simple circuit design, is convenient to manufacture, and easy to install. Said customized integrated electronic circuit attached to the filament-less lamp can replace any lamp device having a transformer leading to reduction in size, thus making it more cost-effective. This invention can be used to sanitize air, water and food to inactivate or kill bacteria, viruses and microbes. It can be installed in any environment such as offices, schools, malls, hotels, airports, and in any indoor space.

Lux is a measure of what proportion of light falls on a surface. But, an attempt to increase the lux in the UV-C lamp more than its capability, can lead to failure of light due to bursting of the filaments. To withstand the intensity of light, the thickness and diameter of the glass tube can be increased. The mercury content can also be increased to improve the life of the UV-C lamp. The customized integrated electronic circuit helps optimize the lux to maximum using the residual mercury until it becomes zero after the light is diffused.

At the Centre for Cellular and Molecular Biology (CCMB), a laboratory under India's Council for Scientific and Industrial Research (CSIR), a test was conducted on a virus sample at 30 watts and 254-nanometer range by using a filament-less UV-C lamp. Alternately, any other lamp with different specifications could be used. The customized integrated electronic circuit can generate 1288 lux in the same lamp as per requirement, thus increasing the thickness of the light. The technology validation results were approved by International Advanced Research Centre for Powder Metallurgy and New Materials (ARCI) while the biological experiments were conducted at CCMB to assess the efficiency of the customized integrated electronic circuit with and without filaments, against SARS-COV-2.

The present invention is a first-of-its-kind technology that excites the electrons and makes electrons move from positive terminal to negative terminal, which increases the voltage to 2000 V by which the tube light glows and immediately drops down to 240 V by maintaining the fluctuation balance.

UV-C lamps that are available in the market have less lux and an attempt to increase the lux in a UV-C lamp available in the market, breaks down filaments leading to failure of the lamp. So, in the present invention, in order to withstand the intensity, both the thickness and diameter of the glass tube are increased, and also, mercury content is optimally utilized until it becomes zero i.e., there will be no residual mercury. The customized integrated electronic circuit increases the life of the UV-C lamp.

The intensity of the UV-C lamp can be customized based on the required sanitization time. The present invention comprising a filament-less UV-C lamp is effective in inactivating or killing SARS-COV-2 virus and other infectious viruses within 15 seconds which is considerably faster than the UV-C lamps or UV-C disinfectant boxes currently available in the market. The technology involves creation of high lux intensity that causes electron excitation thus making the electrons move from one end of the UV-C lamp to another, thereby emitting UV-C light that neutralizes the spike protein present in biological viruses.

Experimental results on lux intensity, time range and efficiency from UV-C lamp with and without filaments are presented in Table 1.

TABLE 1

| | Power | LUX | Sanitization | % |
|---|---|---|---|---|
| Type of Lamp | (Watts) | intensity | time (Sec) | Efficiency |
| Existing/Normal | 30 | 180 LUX | 1200-1800 | 99 |
| UV-C lamp | | | | |
| Filament-less UV- | 30 | 1288 LUX | 15 | 99 |
| C lamp | | | | |

Similar experiments were conducted with normal filaments containing light with choke and starter, and filament-less lamp without choke and without starter. The lux intensity was measured at different wattage. The Power wattage vs. Lux intensity for filament-less lamp without choke and without starter are presented in Table 2.

TABLE 2

| Type of Lamp * | Power (Watts) | LUX intensity |
|---|---|---|
| 1 | 36 | 3250-6700 LUX |
| 2 | 40 | 4000-7600 LUX |
| 3 | 250 | 20000-38000 LUX |

* Filament-less lamp without choke and starter

Irradiance (flux density) measurements were conducted at ARCI. These measurements were done to validate the UV-C lamps with and without the customized electronic circuit connected to the lamps. Irradiance of the UV-C lamps (254 nm) were measured, using a radiometer, at a distance of 5 cm and 10 cm from the source light. Below are the results from UV-C lamp irradiance measurements which were made with and without the customized integrated electronic circuit. The measurements were repeated with the same lamp connected to the customized integrated electronic circuit to observe the increase in the irradiance. The results are presented in Table 3.

TABLE 3

| Type | At 5 cm distance | At 10 cm distance |
|---|---|---|
| Irradiance of 11 W lamp | 80 μW/cm² | 49 μW/cm² |
| (Philips 254 nm) without | | |
| customized integrated | | |
| electronic circuit | | |
| Irradiance of 11 W lamp | 336 μW/cm² | 232 μW/cm² |
| (Philips 254 nm) with | | |
| customized integrated | | |
| electronic circuit | | |

The irradiance values showed that the irradiance increased by approximately 4.8 times when the customized integrated electronic circuit was connected to the lamp. When the customized integrated electronic circuit was not connected, the irradiance values were much less demonstrating the role of the customized electronic circuit.

The dosage can be calculated by using the formula:

$$Dosage ( µJ / {cm}^{2} ) = Irradiance ( µW / {cm}^{2} ) \times Time ( Sec )$$

Without the use of a customized integrated electronic circuit of the present invention, at a distance of 5 cm and irradiance of 80 μW/cm², it would take 62 seconds for germicidal disinfection. However, with the use of the customized integrated electronic circuit of the present invention, at a distance of 5 cm and an irradiance of 336 μW/cm², it would take 20-25 seconds for germicidal disinfection.

The present invention has been tested with SARS-COV-2 virus, maintaining a 30 cm distance between the sample and light source. Experiments were conducted at CCMB, to validate the viral reduction. The lux intensity was maintained at 1288, and the viral concentration at different time points (15, 30, 60, 600, 1200 secs) was measured. The lux intensity was also maintained at 1350, 632 and 405, and the viral concentration at different time points (5, 15, 30, 45, 60, 300 secs) was measured.

For testing, SARS-COV-2 viral isolate (patient origin) was considered as the virus. The host cell line selected was Vero (African green monkey kidney cells (ATCC® CCL-81™). The media and reagents used were Cell Culture Media (Eagle's Minimum Essential Media containing 10% Fetal Bovine Serum), Fetal Bovine Serum (FBS) and Phosphate Buffered Saline (PBS) respectively.

A set of vero cells were grown in DMEM media containing 10% fetal bovine serum (FBS), in a flask. The viral suspension was placed in a quartz cuvette and exposed to the ultra-violet (UV) lamp for specified time (15, 30, 60, 600, 1200 seconds) and (5, 15, 30, 45, 60, 300 seconds). An untreated viral suspension was considered as the control sample. No positive control was used in the study as there is no accepted positive control for SARS-Cov-2. Host cells were infected with viral test sample which were treated with UV for different time periods (15, 30, 60, 600, 1200 seconds) and (5, 15, 30, 45, 60, 300 seconds). The viral test samples and control (untreated) viral samples were incubated for five days. Post-incubation, the viral infectivity in the vero cells was determined. After 5 days of incubation, viral concentrations were determined using reverse transcription polymerase chain reaction (RT-PCR). The assay is schematically presented below.

The virus that was treated upto a time period of 20 minutes was incubated upto five days to study the cytopathogenic effect (CPE).

The TCID50 (Median Tissue Culture Infectious Dose) assay method was used to verify the viral titer of the testing virus.

The percentage (%) survival is determined by cytopathic effects or by viral enumeration in the cell supernatant by Reverse transcription polymerase chain reaction (RT-PCR). The details of the viral concentration determination kit are given below.

The viral Ribonucleic acid (RNA) is isolated from the host cells using the viral Ribonucleic acid (RNA) isolation kit. The various viral Ribonucleic acid (RNA) samples were amplified in Reverse transcription polymerase chain reaction (RT-PCR). The viral particles quantification is based on interpretation using Ct values received from Reverse transcription polymerase chain reaction (RT-PCR).

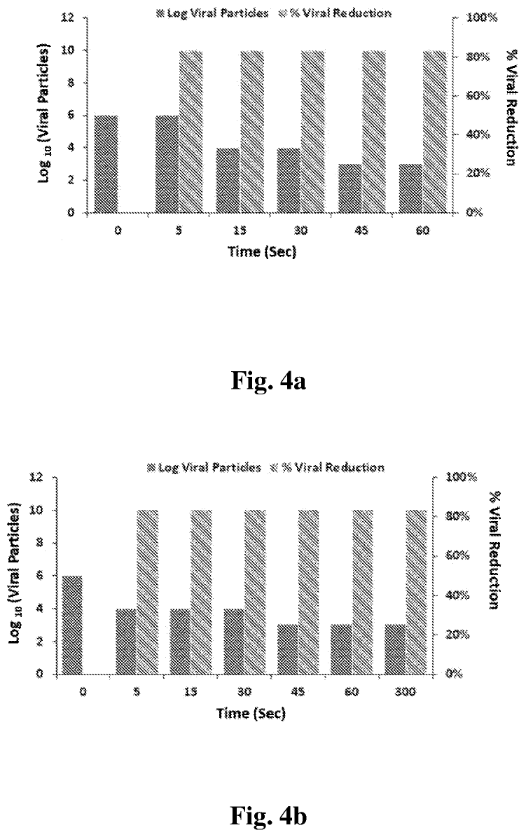

HiGenoMB (Viral RNA Isolation Kit) and MagMAX (Viral/Pathogen II Nucleic Acid Isolation Kit) manufactured by HiMedia and Applied Biosystems were used for viral RNA extraction. Fosun Covid-19 RT-PCR Detection Kit manufactured by Fosun Pharma was used for the assay. The experiments were conducted to validate the viral reduction caused by high lux intensity light from a filament-less lamp with customized integrated electronic circuit. The test results are presented in FIG. 4a and FIG. 4b. The results from the above tests showed a virus reduction of 99% at all the tested time points. The virus number for tested time points reduced from 10^{6.7} to 10^{3.5} at 1288 lux intensity. The virus number for tested time points reduced from 10^{6.6} to 10^{4.1} at 1350 lux intensity. The virus number for tested time points reduced from 10^{6.6} to 10^{4.2} at 632 lux intensity. The virus number for tested time points reduced from 10^{6.6} to 10^{3.6} at 405 lux intensity. The experiment was repeated and the values were averaged to calculate the % virus reduction and presented in Table 4.

TABLE 4

| Tested | Tested time | Lux | Viral number |
|---|---|---|---|
| virus | points (Sec) | intensity | reduction |
| SARS- | 5, 15, 30, 45, 60, 300 | 1350 | 10^{6.6} to 10^{4.1} |
| CoV2 | 5, 15, 30, 45, 60, 300 | 632 | 10^{6.6} to 10^{4.2} |
| | 5, 15, 30, 45, 60, 300 | 405 | 10^{6.6} to 10^{3.6} |
| | 15, 30, 60, 600, 1200 | 1288 | 10^{6.7} to 10^{3.5} |

The regression equation used for measurement of viral particles vs. Ct value of the N-gene specific to SARS-COV2 virus is given below:

(y=−4.9474x+39.723,R²=0.9964) wherein(X=Number of viral particles,y=Ct value)

Number of viral particles are calculated using X=(39.723−Ct_{Ngene @ different time points})/4.9474.

$$( \% Viral reduction ) = \frac{\begin{matrix}Number of viral particles without UV exposure ( control ) - \\ number of viral particles with UV exposure ( test )\end{matrix}}{number of viral particles without UV exposure ( control ) \times 100}$$

According to another embodiment of the present invention also relates to a transformer-less, battery-less device with a customized integrated electronic circuit for generating high density electrons.

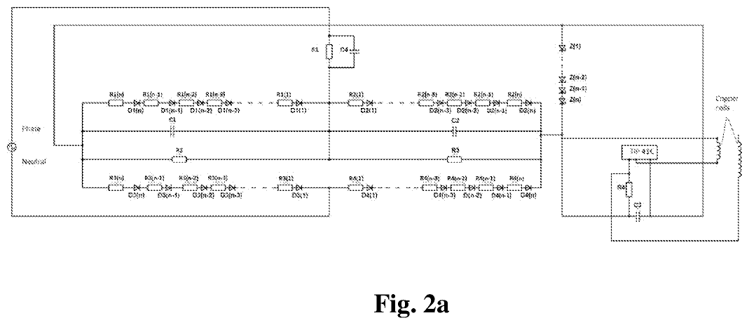

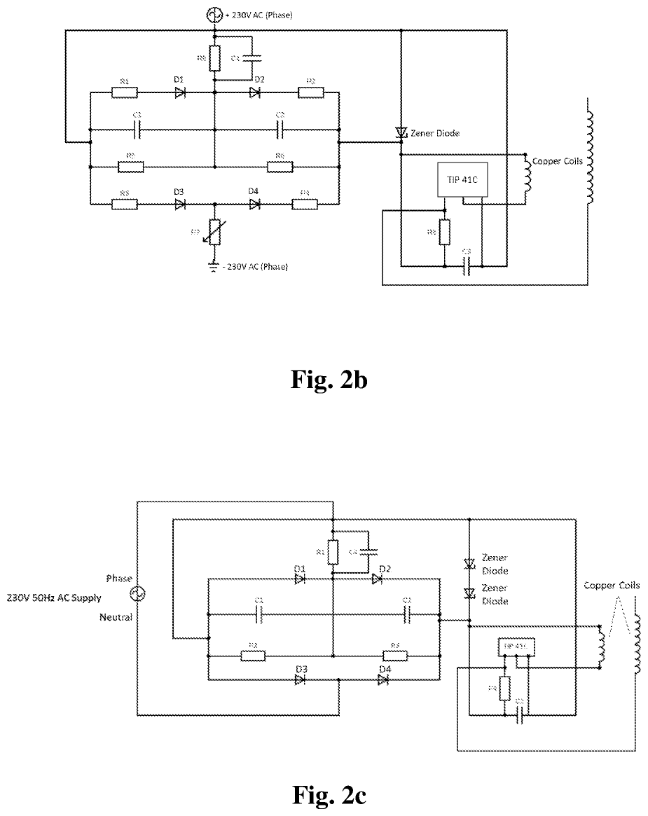

According to an embodiment of the present invention, as shown in FIG. 2a, a transformer-less and battery-less generator with a customized integrated electronic circuit comprises plurality of diodes, plurality of resistors, a variable resistor, capacitors. Zener diodes, a transistor (TIP 41C), and one or more copper coils. The combination of plurality of diodes, and plurality of resistors forms DC bridge rectifier circuit. The combination of plurality of diodes, and capacitors forms a voltage multiplier circuit.

FIG. 2b represents a high-density electron generator circuit that generates high electron acceleration, according to another embodiment of the present invention. The circuit comprises of diodes (D1, D2, D3 and D4), Zener diodes, resistors (R1, R2, R3, R4, R5, R6, R8 and R9), a variable resistor (R7), non-polarized capacitors (C1, C2, C3 and C4), a TIP 41C transistor, and copper coils. The variable resistor R7 employed in the transformer-less and battery-less high-density electron generator with said customized electronic integrated circuit, is made up of Nichrome, Iron, Copper, Aluminium or a combination thereof. The wire gauge thickness, length and number of turns of the copper coil may vary. The values for resistors and capacitors may vary. The size of diodes may also vary. This customized integrated electronic circuit generates high density electrons.

The resistors R1, R2, R3 and R4 have resistance values ranging from 1 ohm to 3 ohms and a power rating of 1000 watt to 2000 watt. The resistors R5 and R6 have 1 mega ohm to 3 mega ohms resistance value and a power rating of 0.25 watts to 5 watts. The variable resistor (R7) has a resistance value ranging from 1 ohm to 9 ohms and a power rating of 500 watts to 2000 watts. It is made up of one metal or a combination of metals. The resistor R8 has a resistance of 1 kilo-ohm to 10 kilo-ohms and a power rating of 1 watt to 5 watts. The Zener diodes have a voltage rating ranging between 9 Volts and 28 Volts. The non-polarized capacitor has a voltage rating ranging from 400 volts to 1050 volts. The transistor used is TIP 41C comprising a base, a collector and emitter terminals. The circuit shown in FIG. 2b has two copper coils, of which, one copper coil has turns ranging between 3 and 9 whereas the second copper coil has turns numbering 900, 9000, 90000, 900000 and so on. The wire gauge of the copper coil, that is, the number of turns, and also the range values for resistors and capacitors, may vary depending upon the size of the coverage area for inactivating/killing pathogens.

According to another embodiment of the present invention, as shown in FIG. 2c, a device with transformer-less and battery-less high-density electron generator along with modified integrated electronic circuit comprises of diodes (D1, D2, D3 and D4), Zener diodes, resistors (R1, R2. R3 and R4), and capacitors (C1. C2, C3 and C4), a transistor (TIP 41C), and copper coils.

The wire gauge thickness, length and number of turns of the copper coil may vary. The values for resistors and capacitors may vary. The size of diodes may also vary. This customized integrated electronic circuit generates high density electrons.

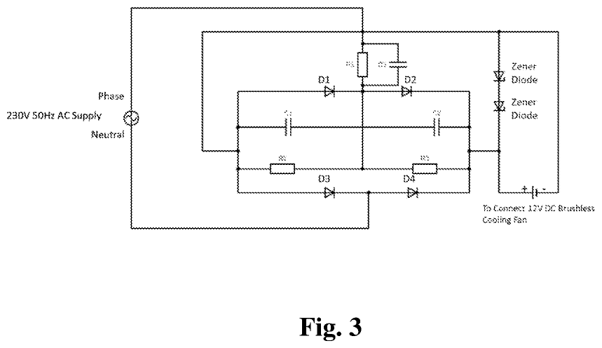

FIG. 3 is a transformer-less and battery-less high-density electron generator provided with a cooling fan. A 12V DC brushless cooling fan is connected to the existing circuit to provide necessary cooling to the Zener diodes. The bridge rectifier circuit converts AC supply into DC supply. The circuit comprises diodes (D1, D2, D3, and D4), resistors (R1, R2, and R3), capacitors (C1, C2 and C3), Zener diodes, and a 12V DC brushless cooling fan. The capacitor C3 is connected across the resistor R1. In the event the device is operated for a long duration of time, the temperature of Zener diodes may increase leading to their failure. In order to protect the Zener diodes from thermal failure, a 12V DC brushless cooling fan is connected.

Said device comprising a transformer-less and battery-less high density electron generator device with a customized integrated electronic circuit, sanitizes surrounding environment when placed on a surface, by inactivating or killing the viruses, bacteria and protozoa. The device of the present invention can inactivate or kill the SARS-COV-2 virus and other infectious viruses within seconds which is many times faster than UV-C lamps currently available in the market for disinfection.

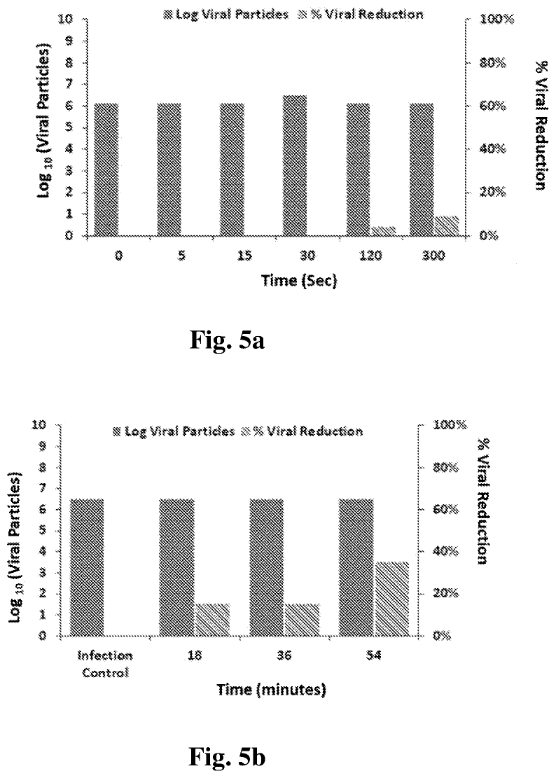

Experiments were conducted at CCMB, to also validate the viral reduction caused by transformer-less and battery-less electron generator with the customized integrated electronic circuit containing copper coil. The distance between the sample and emissions source was maintained at 4 cm. The % viral reduction was calculated by measuring the viral concentration at 5, 15, 30, 120, and 300 seconds respectively. MagMax™ Viral/Pathogen II Nucleic acid Isolation Kit manufactured by Applied Biosystems was used for viral RNA extraction. Fosun Covid-19 RT-PCR Detection Kit manufactured by Fosun Pharma was used for the assay. The device comprising transformer-less and battery-less electron generator with the customized integrated electronic circuit containing copper coil showed 7% viral reduction at 5 minutes. The results obtained from the study are presented in FIG. 5a.

Additional test was also conducted at CCMB to validate the viral reduction caused by transformer-less and battery-less electron generator with the customized integrated electronic circuit containing copper coil. The distance between the sample and emissions source was maintained at 4 cm. The % viral reduction was calculated by measuring the viral concentration at 18, 36 and 54 minutes respectively. MagMax™ Viral/Pathogen II Nucleic acid Isolation Kit manufactured by Applied Biosystems was used for viral RNA extraction. MERIL Covid-19 One-Step RT-PCR Detection Kit manufactured by MERIL Diagnostics was used for the assay. The device comprising transformer-less and battery-less electron generator with the customized integrated electronic circuit containing copper coil showed a viral reduction of 17% at 18 and 36 minutes and 36% at 54 minutes. The results obtained are presented in FIG. 5b. The viral particles reduced from Log 10^{6.6} to Log 10^{6.5}, Log 10^{6.5} to Log 10^{6.4} at 18, 36 and 54 minutes respectively. The experiment was duplicated and the values were averaged to calculate % viral reduction.

The regression equation used for measurement of viral particles Vs Ct value of the N-gene specific to SARS-COV2 virus is given below:

(y=−3.8424x+40.364,R²=0.99) wherein(X=Number of viral particles,y=Ct value)

Number of viral particles are calculated using X=(40.364−Ct_{Ngene @ different time points})/3.8424.

$$( \% Viral reduction ) = \frac{\begin{matrix}Number of viral particles without UV exposure ( control ) - \\ Number of viral particles with UV exposure ( test )\end{matrix}}{Number of viral particles without UV exposure ( control ) \times 100}$$

In accordance with the embodiments of the present invention, the objects of the present invention are achieved through a device for generating high lux intensity light, said device comprising a filament-less lamp with a customized integrated electronic circuit comprising a DC bridge rectifier circuit, a voltage multiplier circuit, a variable resistor, and a strip. Said DC bridge rectifier circuit comprises a combination of a plurality of diodes and resistors, and said voltage multiplier circuit comprises a combination of a plurality of diodes and capacitors. Said filament-less lamp comprising a customized integrated electronic circuit, inactivates or kills pathogens within 15 seconds.

In accordance with the embodiments of the present invention, the objects of the present invention are achieved through a device for generating high density electrons comprising a transformer-less and battery-less generator with a customized integrated electronic circuit comprising a DC bridge rectifier circuit, a voltage multiplier circuit, a variable resistor, plurality of Zener diodes, a transistor (TIP 41C), and one or more copper coils. Said DC bridge rectifier circuit comprises a combination of a plurality of diodes and resistors, and said voltage multiplier circuit comprises a combination of a plurality of diodes and capacitors. Said transformer-less and battery-less generator with a customized integrated electronic circuit generates high density electrons which inactivate or kill pathogens within 18 minutes.

Said device of the various embodiments of the present invention is a lightweight, low cost, high efficiency device with long service life.

It is to be understood, however, that the present invention would not be limited by any means to the components, arrangements and materials that are not specifically described, and any change and modifications to the techniques and approaches can be made without departing from the spirit and scope described in the present invention.

1. A device for generating high lux intensity light comprising:
a filament-less lamp with a customized integrated electronic circuit comprising a combination of plurality of diodes, plurality of resistors, a variable resistor, capacitors; and
a strip attached to the filament-less lamp.

2. The device for generating high lux intensity light according to claim 1, wherein the combination of plurality of diodes and plurality of resistors forms DC bridge rectifier circuit.

3. The device for generating high lux intensity light according to claim 1, wherein the combination of plurality of diodes and capacitors forms a voltage multiplier circuit.

4. The device for generating high lux intensity light according to claim 3, wherein the voltage multiplier circuit enables the conversion of 230 V AC to 650-700 V DC.

5. The device for generating high lux intensity light according to claim 3, wherein the positive terminal of the voltage multiplier circuit is connected to one end of the filament-less lamp through the variable resistor.

6. The device for generating high lux intensity light according to claim 3, wherein the negative terminal of the voltage multiplier circuit is connected to another end of the filament-less lamp.

7. The device for generating high lux intensity light according to claim 1, wherein the filament-less lamp is any one of a failed UV-C lamp, fluorescent lamp, metal halide lamp, mercury vapor lamp, sodium vapor lamp, hydrargyrum medium-arc iodide (HMI) lamp.

8. The device for generating high lux intensity light according to claim 1, wherein the strip attached to the filament-less lamp is made up of silver, copper, gold, aluminum, iron or a combination thereof.

9. The device for generating high lux intensity light according to claim 1, wherein said high lux intensity light effectively inactivates or kills the pathogens.

10. The device for generating high lux intensity light according to claim 9, wherein the pathogens are inactivated or killed within 15 seconds.

11. A device for generating high density electrons comprising:
a transformer-less and battery-less generator with a customized integrated electronic circuit comprising a combination of plurality of diodes, plurality of resistors, a variable resistor, capacitors, and
plurality of Zener diodes, a transistor (TIP 41C), and one or more copper coils.

12. The device for generating high density electrons according to claim 11, wherein the combination of plurality of diodes and plurality of resistors forms DC bridge rectifier circuit.

13. The device for generating high density electrons according to claim 11, wherein the combination of plurality of diodes and capacitors forms a voltage multiplier circuit.

14. The device for generating high density electrons according to claim 13, wherein the voltage multiplier circuit enables the conversion of 230 V AC to 650-700 V DC.

15. The device for generating high density electrons according to claim 11, wherein said high electron generation effectively inactivates or kills the pathogens.

16. The device for generating high density electrons according to claim 11, wherein said high electron generation effectively inactivates or kills the pathogens within 18 minutes.

17. The device for generating high density electrons according to claim 11, wherein a cooling fan is connected to the circuit.

18. A method of inactivating or killing pathogens, comprising the method of exposing a surface to a device comprising of:
a filament-less lamp with a customized integrated electronic circuit for generating high lux intensity light, wherein
said integrated electronic circuit comprises a combination of plurality of diodes, plurality of resistors, a variable resistor, capacitors, and a strip attached to the filament-less lamp.

19. A method of inactivating or killing pathogens, comprising the method of exposing a surface to a device comprising of:
a transformer-less and battery-less generator with a customized integrated electronic circuit for generating high density electrons, wherein
said integrated electronic circuit comprises a combination of plurality of diodes, plurality of resistors, a variable resistor, capacitors, plurality of Zener diodes, a transistor (TIP 41C), and one or more copper coils.

20. Use of a device in inactivating or killing pathogens in surrounding environment, wherein said device comprises of:
a filament-less lamp with a customized integrated electronic circuit for generating high lux intensity light, wherein
said integrated electronic circuit comprises a combination of plurality of diodes, plurality of resistors, a variable resistor, capacitors, and a strip attached to the filament-less lamp.

21. Use of a device in inactivating or killing pathogens in surrounding environment, wherein said device comprises of:
a transformer-less and battery-less generator with a customized integrated electronic circuit for generating high density electrons, wherein
said integrated electronic circuit comprises a combination of plurality of diodes, plurality of resistors, a variable resistor, capacitors, plurality of Zener diodes, a transistor (TIP 41C), and one or more copper coils.
